(19) United States
(12) Patent Application Publication
(10) Pub. No.: US 2026/0335595 A1
(43) Pub. Date: Oct. 29, 2026

(54) CATHETER GATHERING AND DEPLOYMENT DEVICE

(71) Applicant: BOARD OF REGENTS OF THE UNIVERSITY OF NEBRASKA, Lincoln, NE (US)

(72) Inventors: Daniel Surdell, Omaha, NE (US); Alaric Schiltz, Gretna, NE (US); Mike Altidor, Lincoln, NE (US); Sangjin Ryu, Lincoln, NE (US); Jacob Green, Lincoln, NE (US); Bethany Lowndes, Omaha, NE (US)

(21) Appl. No.: 19/657,613

(22) Filed: Apr. 24, 2026

Related U.S. Application Data
(60) Provisional application No. 63/796,473, filed on Apr. 29, 2025.

(51) Int. Cl. A61M 25/00; A61M 25/06

(52) U.S. Cl. CPC A61M 25/0074; A61M 2025/0006; A61M 2025/0042; A61M 2025/0681; A61M 2210/12

(57) ABSTRACT

A deployment assembly may include a housing configured to fit within a lumen of a catheter, the housing comprising: a seating portion, configured to partially seat an elongate member; and a base portion opposite the seating portion. The deployment assembly may include a clamp element disposed between the seating portion and the base portion, and configured to translate between the seating portion and the base portion. The deployment assembly may include a compressor plate coupled to a base side of the clamp element. The deployment assembly may include a biasing element operatively coupled to the compressor plate and the base portion and configured to bias the compressor plate toward the seating portion, wherein biasing the compressor plate toward the seating portion causes the clamp element to translate to a closed configuration that prevents the elongate member from sliding axially through the catheter.

### CROSS-REFERENCE TO RELATED APPLICATION

The present application claims the benefit under 35 U.S.C. § 119(e) of U.S. Provisional Application Ser. No. 63/796,473, filed Apr. 29, 2025, which is incorporated herein by reference in the entirety.

### GOVERNMENT SUPPORT

This invention was developed with U.S. government support under U54 GM115458 awarded by the National Institutes of Health. The U.S. government has certain rights in this invention.

### BACKGROUND

Catheters have emerged as a critical tool in the endovascular treatment of brain aneurysms. Microcatheters, characterized by their small diameter and high flexibility, enable precise navigation through the intricate vascular network of the brain. Microcatheters may facilitate the delivery of therapeutic agents, such as coils and flow diverters, directly to the aneurysm site. This targeted approach of using microcatheters may not only minimize damage to surrounding tissues but also enhance the efficacy of the treatment. However, a single catheter sometimes does not provide the surgeon with enough control for precise coil placement or more complex situations, requiring the use of multiple catheters. Organizing, controlling, and activating multiple microcatheters once deployed has been a challenge for the field. Therefore, there is a need for systems and methods for controlling multiple catheters and/or microcatheters during a medical procedure.

### SUMMARY

In an aspect, a deployment assembly for a distal end of a catheter is disclosed. In one or more embodiments, the deployment assembly includes: a housing configured to fit within a lumen of the catheter, the housing including: a seating portion, configured to partially seat an elongate member; and a base portion opposite the seating portion. In one or more embodiments, the deployment assembly includes a clamp element disposed between the seating portion and the base portion and configured to translate between the seating portion and the base portion. In one or more embodiments, the deployment assembly includes a compressor plate coupled to a base side of the clamp element, and a biasing element operatively coupled to the compressor plate and the base portion and configured to bias the compressor plate toward the seating portion, wherein biasing the compressor plate toward the seating portion causes the clamp element to translate to a closed configuration that prevents the elongate member from sliding axially through the catheter.

In another aspect, a system is disclosed. In one or more embodiments, the system includes a catheter, an elongate member configured to translate axially relative to the catheter, and a deployment assembly disposed at a distal end of the catheter and configured to control axial movement of the elongate member relative to the catheter. In one or more embodiments, the deployment assembly includes: a housing configured to fit within a lumen of the catheter, the housing including: a seating portion, configured to partially seat an elongate member; and a base portion opposite the seating portion. In one or more embodiments, the deployment assembly includes a clamp element disposed between the seating portion and the base portion, and configured to translate between the seating portion and the base portion. In one or more embodiments, the deployment assembly includes a compressor plate coupled to a base side of the clamp element, and a biasing element operatively coupled to the compressor plate and the base portion and configured to bias the compressor plate toward the seating portion, wherein biasing the compressor plate toward the seating portion causes the clamp element to translate to a closed configuration that prevents the elongate member from sliding axially through the catheter.

In another aspect, a method for controlling an elongate member during a medical procedure is disclosed. In one or more embodiments, the method includes advancing a catheter through a vessel toward a treatment area, wherein the catheter includes: the elongate member; and a deployment assembly positioned on a distal end of the catheter, wherein the deployment assembly is configured in a closed configuration, wherein the elongate member is restricted from translating axially through the catheter when the deployment assembly is configured in the closed configuration. In one or more embodiments, the method includes maintaining the catheter at a position adjacent to the treatment area. In one or more embodiments, the method includes actuating a tensile element, wherein actuating the tensile element counteracts a biasing force of a biasing element in the deployment assembly and causes the deployment assembly to transition from the closed configuration to an open configuration, wherein the elongate member is configured to translate axially through the catheter when the deployment assembly is configured in the open configuration. In one or more embodiments, the method includes translating the elongate member relative to the catheter.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

The numerous advantages of the disclosure may be better understood by those skilled in the art by reference to the accompanying figures.

FIG. 1A illustrates a perspective front view of a distal end of a deployment assembly positioned within a lumen of a catheter, in accordance with one or more embodiments of the disclosure.

FIG. 1B illustrates a perspective side view of the deployment assembly in the closed configuration, in accordance with one or more embodiments of the disclosure.

FIG. 1C illustrates a semi-transparent perspective front view of the deployment assembly, in accordance with one or more embodiments of the disclosure.

FIG. 1D illustrates a perspective exploded view of the deployment assembly, in accordance with one or more embodiments of the disclosure.

FIG. 2 illustrates a process-flow diagram depicting a method for operating the deployment assembly, in accordance with one or more embodiments of the disclosure.

FIG. 3 illustrates a process-flow diagram depicting a method for controlling an elongate member during a medical procedure, in accordance with one or more embodiments of the disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the present disclosure.

As used herein, a letter following a reference numeral is intended to reference an embodiment of the feature or element that may be similar, but not necessarily identical, to a previously described element or feature bearing the same reference numeral (e.g., 1, 1a, 1b). Such shorthand notations are used for purposes of convenience only and should not be construed to limit the disclosure in any way unless expressly stated to the contrary.

Finally, as used herein, any reference to “one embodiment” or “some embodiments” means that a particular element, feature, structure, or characteristic described in connection with the embodiment is included in at least one embodiment disclosed herein. The appearances of the phrase “in some embodiments” in various places in the specification are not necessarily all referring to the same embodiment, and embodiments may include one or more of the features expressly described or inherently present herein, or any combination of sub-combination of two or more such features, along with any other features which may not necessarily be expressly described or inherently present in the instant disclosure.

A deployment assembly for a distal end of a catheter is disclosed. The deployment assembly includes a housing configured to fit within a lumen of a catheter, such as a guide catheter, that includes a seating member configured to partially or wholly seat an elongate member, such as a microcatheter or a snare device, and a clamping assembly that biases a compressor plate towards the seating member, preventing the elongate member from sliding axially through the catheter. The deployment assembly includes or is coupled to a tensioning wire or thread that, when pulled, releases the elongate member from the clamping assembly, enabling the elongate member to slide relative to the catheter and out of the distal end of the catheter, allowing a controlled delivery of microcatheters or other tools to the treatment site.

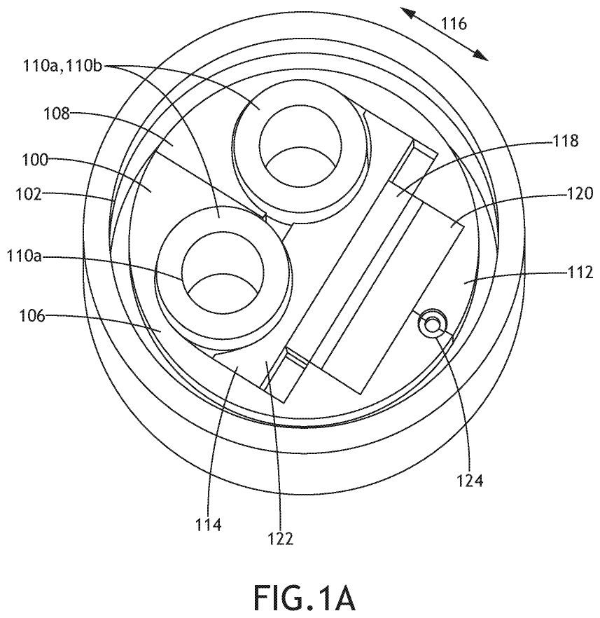

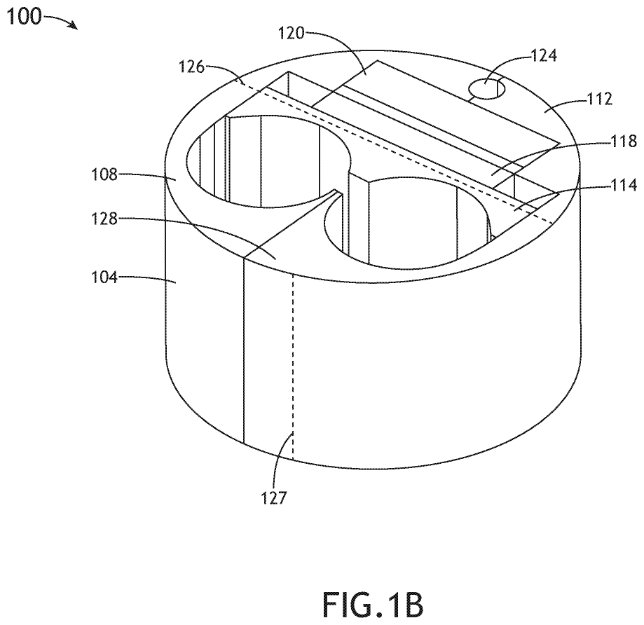

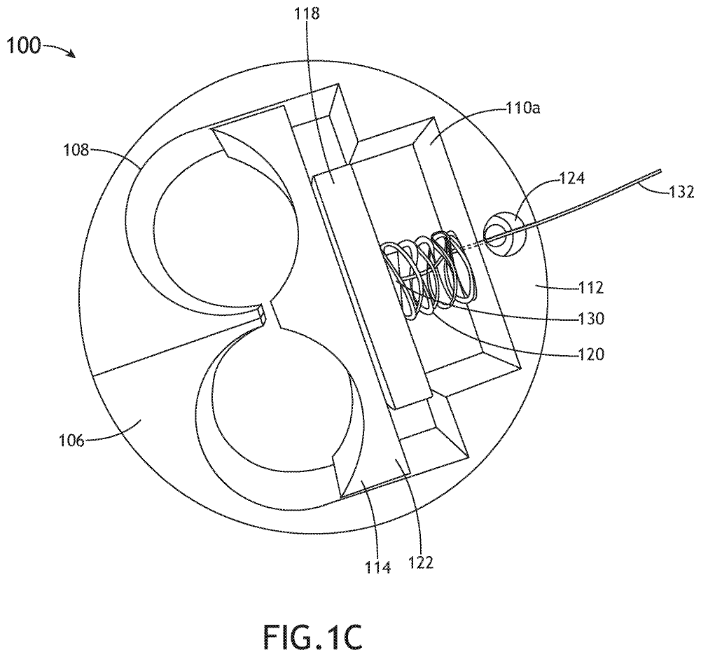

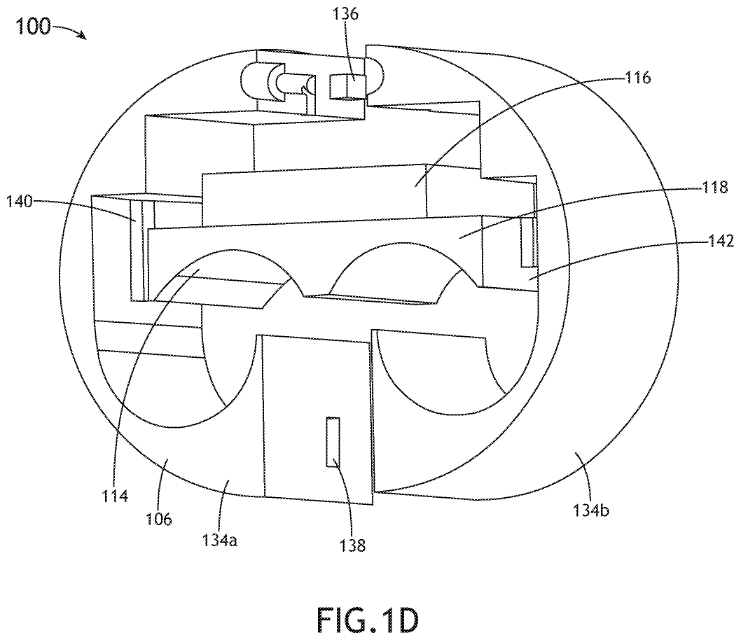

FIG. 1A illustrates a perspective front view of a distal end of a deployment assembly 100 positioned within a lumen 102 of a catheter 104 (e.g., a guide catheter), in accordance with one or more embodiments of the disclosure. The deployment assembly 100 may be utilized by one or more types of medical catheters 104, including, but not limited to, vascular catheters, cardiac catheters, urinary catheters, respiratory catheters, neurological catheters, and gastrointestinal catheters. For example, the catheter 104 may include a vascular catheter designed for the repair of aneurysms (e.g., via coil delivery).

In embodiments, the deployment assembly 100 includes a housing 106, such as a collar housing, which fits within the lumen 102 of the catheter 104. The housing 106 may be formed of any suitable biocompatible material, such as a thermoplastic. For example, the housing 106 may be formed of ultra-high molecular weight polyethylene (UHMW-PE). Other components of the deployment assembly 100 may be formed of similar materials, such as polylactic acid (PLA). One or more of the components of the deployment assembly 100 may be formed via additive manufacturing methods.

In embodiments, the housing 106 includes a seating portion 108 configured to at least partially seat (e.g., nest or cradle) one or more elongate member 110a, 110b, such as a microcatheter. For example, the seating portion 108 may be configured to seat one or more microcatheters, such as microcatheters for delivering coils for aneurysm repair. The elongate member may include one or more other tools besides a microcatheter, including, but not limited to, a snare device, a thrombectomy device, and an imaging tool. The housing 106 may further include a base portion 112 opposite of, or near opposite of, the seating portion 108.

In embodiments, the deployment assembly 100 includes a clamp element 114 disposed between the seating portion 108 and the base portion 112, and configured to translate between the seating portion 108 and the base portion 112 (e.g., via a reciprocating movement 116). The position of the clamp element 114 after translation may define an open configuration or a closed configuration. For example, in an open configuration, the clamp element 114 has translated away from the seating portion 108, allowing one or more elongate members 110a, 110b to move axially (e.g., slide) relative to the catheter 104. In another example, in a closed configuration the clamp element 114 has translated towards the seating portion 108, securing one or more elongate members 110a, 110b to the seating portion 108 via compression.

In embodiments, the deployment assembly 100 includes a compressor plate 118 and a biasing element 120. The compressor plate 118 may be coupled to a base side 122 of the clamp element 114, and the biasing element 120 may be coupled (e.g., operatively coupled) to the base portion 112 and configured to bias against the compressor plate 118. For example, the biasing element 120 may be configured to bias the compressor plate 118 toward the seating portion 108. Biasing the compressor plate 118 may then cause the clamp element 114 to translate from the open configuration to the closed configuration. The compressor plate 118 distributes the force of the biasing element 120 and assists in constraining motion. The biasing element 120 may include any type of biasing mechanism, including, but not limited to, a spring, such as a compression spring.

In embodiments, one or more of the clamp element 114, the compressor plate 118, and the biasing element 120 may be merged together within one body. For example, clamp element 114 and the compressor plate 118 may be merged into a single body. In another example, the compressor plate 118 and the biasing element 120 may be merged into a single body. In another example, the clamp element 114, the compressor plate 118, and the biasing element 120 are merged into a single body.

In embodiments, the deployment assembly 100 includes an aperture 124 configured to allow passage of a tensile element (e.g., a tension wire or thread) to one or more of the clamp element 114, the compressor plate 118, and/or the biasing element 120. Once coupled to the clamp element 114, the compressor plate 118, and/or the biasing element 120, the tensile element may be pulled, or otherwise actuated, counteracting the biasing force of the biasing element 120 and causing the clamp element 114 to translate from the closed configuration to the open configuration.

FIG. 1B illustrates a perspective side view of the deployment assembly 100 in the closed configuration, in accordance with one or more embodiments of the disclosure. In embodiments, a diameter 126 along a distal face 128 of the deployment assembly 100 ranges from 1.3 mm to 4.0 mm (e.g., 4-12 Fr). For example, the diameter may range from 1.3 mm to 3.3 mm (e.g., 4-10 Fr). In another example, the diameter may range from 1.3 mm to 2.7 mm (e.g., 4-8 Fr). In another example, the diameter may range from 1.3 mm to 2.0 mm (e.g., 4-6 Fr). In another example, the diameter may range from 1.3 mm to 1.7 mm (e.g., 4-5 Fr). For instance, the diameter may be approximately or substantially 1.5 mm. The deployment assembly 100 may have a depth 127 of approximately 1 mm.

FIG. 1C illustrates a semi-transparent perspective front view of the deployment assembly 100, in accordance with one or more embodiments of the disclosure. The biasing element 120 is shown as a compression spring.

In embodiments, the deployment assembly 100 includes a retention element 130 disposed on at least one of the clamp element 114, the compressor plate 118 or the biasing element 120 couplable to the tensile element 132. The retention element 130 may include any type of fastening technology (e.g., a coupler), including, but not limited to, a staple, an anchor, a hook, and a screw.

FIG. 1D illustrates a perspective exploded view of the deployment assembly 100, in accordance with one or more embodiments of the disclosure. The view illustrates the clamp element 114 and the compressor plate 118, omitting the biasing element 120.

In embodiments, the housing 106 is formed of two or more housing components 134a, 134b that are separately formed, then bonded together. For example, the housing components 134a, 134b may include shaft 136 and hole 138 structures that connect to complementary hole and shaft structures that bind the housing components 134a, 134b together, such as with a friction fit and/or interference fit. The housing components 134a, 134b, as well as other components of the deployment assembly 100, may be adhered to each other via an adhesive, such as via a cyanoacrylate glue and/or an epoxy.

In embodiments, the translation of the clamp element 114 is guided via one or more tracks 140 (e.g., grooves) disposed on an inner surface of the housing 106. For example, the sides of the clamp element 114 may include track-engaging members 142, (e.g., lateral ends such as tongues), that fit within or otherwise engage the track, enabling the clamp element 114 to be secured within the housing 106 while still being translatable.

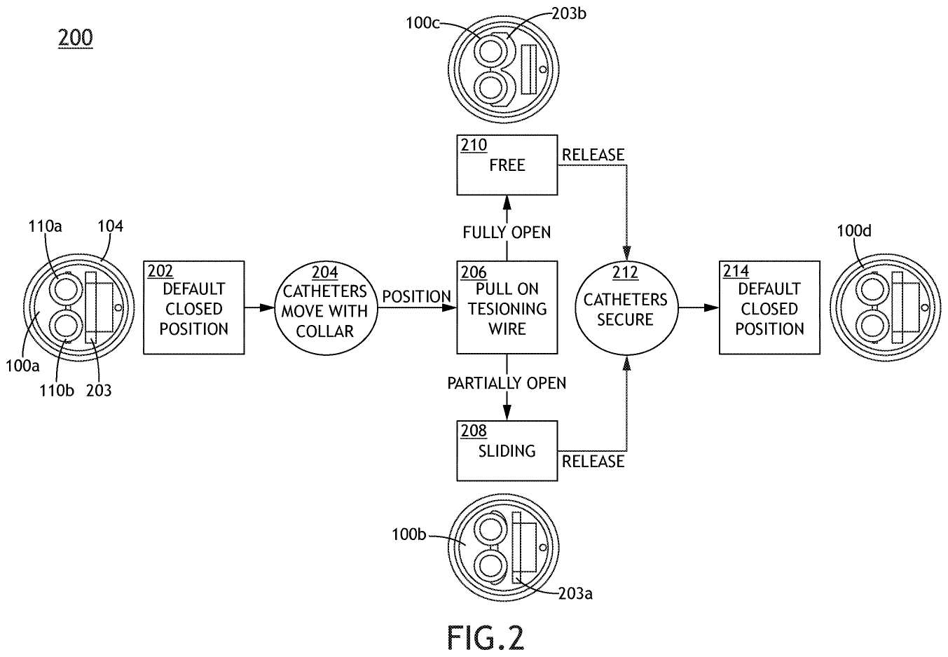

FIG. 2 illustrates a process-flow diagram depicting a method 200 for operating the deployment assembly 100, in accordance with one or more embodiments of the disclosure. The method 200 may be performed during a medical procedure, such as a coil embolization, where elongate members 110a, 110b (e.g., microcatheters) are to be employed. In embodiments, the method 200 includes a step 202 where the deployment assembly 100 is in the default or closed configuration (e.g., the clamp element 114 may be biased to a closed configuration unless the tensile element 132 is actuated). The closed configuration may be indicated by the large space 203 between the clamp element 114 and the base portion 112, as indicated in deployment assembly 100a.

In embodiments, the method 200 includes a step 204, where the guide catheter 104 moves with the deployment assembly 100. For example, while the deployment assembly 100 is in the closed configuration, the deployment assembly 100 is moved via the guide catheter 104 to a position near the treatment site. The elongate members 110a, 110b stay in place due to being secured by the deployment assembly 100 in the closed configuration.

In embodiments, the method 200 includes a step 206, where the tensile element 132 (e.g., a tensioning wire) is pulled. The pulling of the tensioning wire fully or partially frees the elongate members 110a, 110b from the seating portion 108, allowing the method 200 to progress to a step 208 of sliding the elongate members 110a, 110b relative to the deployment assembly 100 (e.g., when the deployment assembly 100 is in a partially opened configuration), or a step 210 of freeing the elongate members 110a, 110b (e.g., when the deployment assembly 100 is in a fully open configuration). The partially opened configuration is shown as the deployment assembly 100 having a slightly smaller space 203a between the clamp element 114 and the base portion 112, as indicated in deployment assembly 100b. The fully opened configuration is shown as the deployment assembly 100 having a space 203b between the seating portion 108 and the base portion 112, as indicated in deployment assembly 100c.

In embodiments, the method 200 includes a step 212 of securing the elongate members 110a, 110b. For example, by releasing the tensile element 132, the biasing element 120 then biases the compressor plate 118, causing the translation of the clamp element 114 from the open configuration to the default closed configuration 214, as shown by deployment assembly 100d, which is similar to deployment assembly 100a.

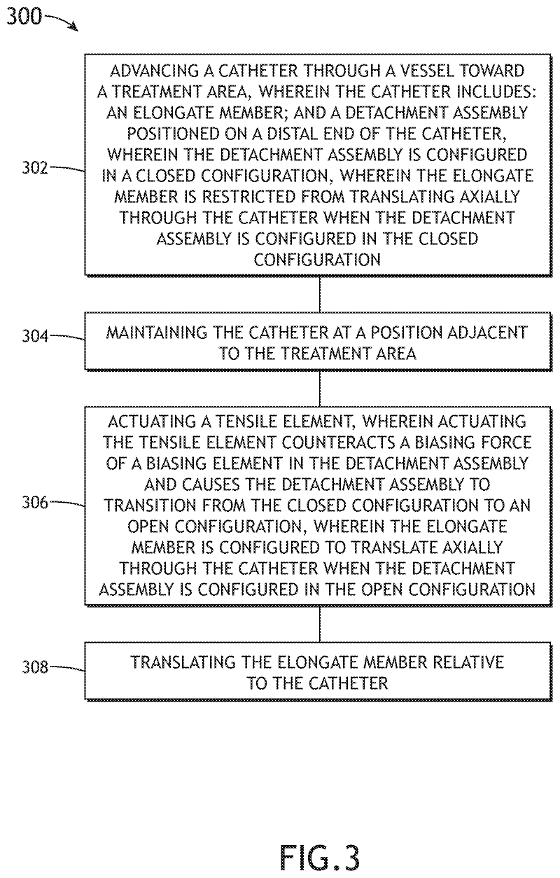

FIG. 3 illustrates a process-flow diagram depicting a method 300 for controlling an elongate member 110a, 110b (e.g., one or more microcatheters) during a medical procedure, in accordance with one or more embodiments of the disclosure. The method 300 may include utilizing the deployment assembly 100 during a coil embolization or other catheter-related medical procedure, as described herein.

In embodiments, the method 300 includes a step 302 of advancing a catheter 104 through a vessel toward a treatment area, wherein the catheter 104 includes: the elongate member 110a, 110b; and a deployment assembly 100 positioned on a distal end of the catheter 104, wherein the deployment assembly 100 is configured in a closed configuration, wherein the elongate member 110a, 110b is restricted from translating axially through the catheter 104 when the deployment assembly 100 is configured in the closed configuration.

In embodiments, the method 300 includes a step 304 of maintaining the catheter 104 at a position adjacent to the treatment area. For example, the catheter 104, once having arrived at the treatment area, may still move slightly back and forth at the treatment area during the procedure while still being maintained adjacent to the treatment area. The catheter 104 may be considered adjacent to the treatment area even if a small width of the vessel blocks the advancement of the catheter 104 immediately upon the treatment area.

In embodiments, the method 300 includes a step 306 of actuating a tensile element 132 (e.g., a tension wire or thread), wherein actuating the tensile element 132 counteracts a biasing force of a biasing element 120 in the deployment assembly 100 and causes the deployment assembly 100 to transition from the closed configuration to an open configuration, wherein the elongate member 110a, 110b is configured to translate axially through the catheter 104 (e.g., begins protruding distally from the catheter 104) when the deployment assembly 100 is configured in the open configuration. The method 300 further includes a step 308 of translating the elongate member 110a, 110b relative to the catheter 104 (e.g., the elongate member 110a, 110b begins protruding distally from the catheter 104). The translation of the elongate member 110a, 110b may indicate an active part of the procedure. For example, in a coil embolization, once the elongate members 110a, 110b (e.g., the microcatheters) have begun protruding from the catheter 104, they may localize to the target aneurysm and begin filling the aneurysm cavity with coils.

Once the active part of the procedure is finished, the method 300 may include a step of releasing the tensile element 132, wherein releasing the tensile element 132 causes the deployment assembly to transition from the open configuration to the closed configuration. The method 300 may also include a step of removing the catheter 104 from the vessel.

### EXAMPLES

The following examples are provided to further describe and enable the subject matter disclosed herein and to illustrate representative implementations, experimental protocols, and/or performance results. Unless expressly stated otherwise, the examples are not intended to limit the scope of the appended claims or to define any particular embodiment as essential. Any terminology, headings, figure captions, references, or stylistic conventions appearing in such manuscript content are presented for convenience and readability and shall not be construed to narrow the meaning of any claim term unless the claim term is expressly defined herein.

### Example 1: Design of a Deployment Assembly for a Distal End of a Catheter

Intracranial aneurysms occur in blood vessels in the brain. An aneurysm is a ballooned area of a blood vessel caused by a weakened vessel wall. To repair aneurysms in the brain, one of the most common procedures is called endovascular coiling. Endovascular coiling involves inserting a catheter system through blood vessels to reach the site of the aneurysm. Generally, a larger diameter guide catheter is inserted first until it is too large to continue. Microcatheters of a smaller diameter are fed through the guide catheter and past it to reach the aneurysm site.

Once the catheters have reached the aneurysm, embolization coils are placed into the catheter and used to occlude the aneurysm. This technique is successful if the embolization coils fully occlude the aneurysm, stopping blood from entering the site.

When surgeons perform aneurysm repair procedures, multiple instruments are utilized in addition to the endovascular coils. Depending on the exact situation, tools like stents and balloons will be necessary for the procedure to be successful. The use of multiple tools simultaneously is a necessary component in many of these procedures. When multiple tools are used at the same time, multiple microcatheters must be used.

When surgeons perform aneurysm repair procedures, the control of microcatheters within the blood vessels can be cumbersome as the catheters must be controlled at the proximal end. Especially in cases where multiple instruments are utilized and multiple microcatheters are necessary, instruments and lumens can become intertwined or interact negatively with one another. Therefore, there is a need for a method and device for gaining better control of multiple lumens. This project's aim was to focus on a deployment mechanism for the catheters'distal ends.

The specific objective is to develop a minimum 3 cm (about 1.18 in) long separation mechanism at the double lumen microcatheters'distal ends. The separation is sufficiently long to provide the branches of the two lumens with the ability to interact as the coils are inserted into the aneurysm.

The constraints of this project are dependent primarily on the size of the implementation. Because the device must travel through the human vascular system and reach specific parts of the brain; the deployment mechanism cannot add significantly to the size of normally utilized catheters. More specifically, the device cannot be larger than 1.5 mm (about 0.06 in) in diameter.

Another dimension requirement is the length of the device from the user to aneurysm. The device will typically be inserted near the groin into the femoral artery and then travel through blood vessels into the brain. The length of this pathway is about 160-165 cm (about 5.41 ft). Thus, the device must be able to be operated at that distance away from the user.

Designing a medical device means it must be reliable and must not be damaging to the body. This means the device cannot damage the arterial walls or leave particulate matter in the bloodstream. The deployment device may have a reliability greater than 95% when finalized. These criteria will produce successful and repeatable medical procedures with little worry about detrimental effects caused by the deployment process. Furthermore, any design must be compatible with common instrumentation like coils, stents, and balloons.

The Collar-Clamp design is like the Collar-Air Bladder. The microcatheters may be inside the collar during operation. In this case, the microcatheters may be secured using a spring force and a moving clamp. This moving clamp would push the microcatheters into a bottom clamp, thus securing the catheters. When wanting to detach, the operator pulls on a tensioning wire to move the clamp up and compress the spring. To secure the catheters, the operator would let go of the tension wire by moving the clamp back to its default position.

The collar and catheters would be designed to move freely within the guide catheter and out of its distal end. This means the collar could travel anywhere with the microcatheters, unlike the air bladder design. In an analysis of the distribution of forces in the Collar-Clamp design, the force would be distributed along the outer surface of the microcatheters and acting radially towards the center because the device would contact the collar on all sides. Below are the equations used in this analysis:

σ_{spring}«σ_{yield} (1)

σ_{yield}=F_{yield}/Aₜₒₜ⇒F_{yield}=σ_{yield}·Aₜₒₜ (2)

F_{yield}=kΔI⇒k=F_{yield}/ΔI. (3).

Equation 1 describes the requirement for only elastic deformation to occur: the yield stress of the microcatheters must be significantly greater than the stress on the microcatheters created by the spring. The yield stress for polyurethane, a common material for making microcatheters, is 4 MPa. From this, the force produced at the yield stress is found to be 2204 N in Equation 2.

The found force can be used to find the spring constant required of the spring. For the microcatheters to fit in the collar, the spring must be compressed by 0.2149 mm. Using the yield force and the distance, the spring constant is 10.4×10^6 N/m. So long as the real spring constant is below the calculated spring constant, the microcatheters will not deform plastically.

Compression equations are difficult to utilize, yield inaccurate results, and are often unnecessary. Materials in compression have a better ability to resist deformation than in tension because the atoms are being pushed into each other rather than split apart. If there is success in analyzing tension, then success can be expected when in compression.

F_{friction}=μF_{yield} (4)

There is a desire to have friction in the system when moving the collar with the catheters. There should be no slipping between the collar and catheters because this could create obstacles during the procedure. However, too much friction has its detriments because slipping is desired when the collar is open.

The friction coefficient was chosen from a friction test between nitrile and knurled steel to be an average of 0.425. If the Moving Top Collar of the clamp was coated in nitrile, then we expect a higher coefficient of friction between nitrile and polyurethane. The yield force was chosen from the previous section. With these two factors, the force of friction between the microcatheter and the Moving Top Clamp is 952 N.

A major necessary analysis was to determine whether the collar device was able to withstand blood pressure. In this finite element analysis (FEA), the collar housing was analyzed when it is subjected to the maximum possible blood pressure. The analysis was completed with a pressure of 370 mmHg applied to the outer surface of the collar housing. The goal of this analysis was to determine if the collar has acceptable strength when subjected to blood pressure. By completing the analysis, the team could determine whether the device is strong enough to withstand the bioenvironment within blood vessels without significant deformation or fracture.

To perform the analysis, a prospective material had to be chosen for the collar housing. The team researched commonly used biocompatible materials, mostly thermoplastics. After careful consideration, ultra-high molecular weight polyethylene (UHMW-PE) was chosen as the material for the collar housing. UHMW-PE is biocompatible, which means that it will not cause ill effects to bodily tissues, and it is viable for injection molding. With the material and pressure identified, the FEA and a factor of safety (FOS) plot was created in SolidWorks. The FOS of the collar is strictly due to blood pressure forces and is at a minimum value of approximately 700. This means that the collar has more than enough strength to withstand the forces of blood pressure.

The Collar-Clamp design is composed of 6 components. The Collar Housing (e.g., housing 106), which also serves as a bottom clamp, separates into two parts. The two parts are guided together using slots and keys. This ensures the Tension Wire Hole is correctly aligned. The two microcatheters (e.g., elongate members 110a, 110b) will sit at the bottom of the Collar Housing in the two U-shaped cavities (e.g., seating portion 108).

At the top of the Collar Housing, the Spring Applicator (e.g., biasing element 120) will be mounted on the ceiling, and the Compressor Plate (e.g., compressor plate 118) will be mounted at the bottom of the Spring Applicator. The tensioning wire will be fed through the Tension Wire Hole (e.g., aperture 124), Spring Applicator, and mounted onto the Compressor Plate. The purpose of the compressor plate is to distribute the force of the spring and help constrain motion.

The Moving Top Clamp (e.g., clamp element 114) will be mounted onto the bottom of the Compressor Plate and placed in two tracks on each side of the Collar Housing. The tracks will help guide the motion of the clamp and ensure it can move smoothly. Multiple 3D printing techniques were used to create the custom parts. The compressor plates were printed out of black PLA plastic.

The prototypes also utilized multiple off-the-shelf components in their assembly. The devices were joined together using cyanoacrylate glue and quick-setting epoxy. These adhesives were used to join the two halves of the housing together and to join the tension wire to the Compressor Plate. Similarly, the Compressor Plate was adhered to the back of the Moving Top Clamp. The spring is also a standard off-the-shelf component. The wave spring was chosen based on its size and form factor. A variety of springs with different stiffnesses were purchased and once the 3D-printed parts were available and assembly began, one was chosen to best fit the model.

The herein-described subject matter sometimes illustrates different components contained within or connected with other components. It should be understood that the depicted architectures are merely exemplary and that many other architectures can be implemented to achieve the same functionality. In a conceptual sense, any arrangement of components that achieves the same functionality is effectively “associated” with the desired functionality. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims.

What is claimed:

1. A deployment assembly for a distal end of a catheter comprising:
a housing configured to fit within a lumen of the catheter, the housing comprising:
a seating portion, configured to partially seat an elongate member; and
a base portion opposite the seating portion;
a clamp element disposed between the seating portion and the base portion, and configured to translate between the seating portion and the base portion;
a compressor plate coupled to a base side of the clamp element; and
a biasing element operatively coupled to the compressor plate and the base portion and configured to bias the compressor plate toward the seating portion, wherein biasing the compressor plate toward the seating portion causes the clamp element to translate to a closed configuration that prevents the elongate member from sliding axially through the catheter.

2. The deployment assembly of claim 1, further comprising a retention element disposed upon at least one of the clamp element, the compressor plate, and the biasing element, wherein the retention element is couplable to a tensile element, wherein an actuation of the tensile element counteracts a biasing force of the biasing element and causes the clamp element to translate to an open configuration that allows the elongate member to slide through the catheter.

3. The deployment assembly of claim 2, wherein the housing further comprises an aperture configured to allow passage of the tensile element to the retention element.

4. The deployment assembly of claim 2, wherein the seating portion is configured to seat two elongate members.

5. The deployment assembly of claim 4, wherein at least one of the two elongate members comprises a microcatheter.

6. The deployment assembly of claim 4, wherein the housing comprises a biocompatible thermoplastic material.

7. The deployment assembly of claim 6, wherein the housing comprises ultra-high molecular weight polyethylene.

8. The deployment assembly of claim 1, wherein the housing comprises tracks disposed radially along an inner surface of the housing between the seating portion and the base portion, wherein the clamp element comprises track-engaging members on lateral ends that engage the tracks, wherein the tracks guide the track-engaging members as the clamp element translates from an open configuration to a closed configuration.

9. The deployment assembly of claim 1, wherein the biasing element comprises a compression spring.

10. The deployment assembly of claim 2, wherein the retention element comprises a coupler that couples the tensile element to at least one of the clamp element, the compressor plate, and the biasing element.

11. The deployment assembly of claim 1, wherein the biasing element is disposed between the compressor plate and the base portion.

12. The deployment assembly of claim 2, wherein the clamp element is biased to a closed configuration unless the tensile element is actuated.

13. The deployment assembly of claim 1, wherein the housing comprises a distal face having a diameter ranging from 1.3 mm to 1.7 mm.

14. A system comprising:
a catheter;
an elongate member configured to translate axially relative to the catheter; and
a deployment assembly disposed at a distal end of the catheter and configured to control axial movement of the elongate member relative to the catheter, the deployment assembly comprising:
a housing configured to fit within a lumen of the catheter, the housing comprising:
a seating portion, configured to partially seat the elongate member; and
a base portion opposite the seating portion;
a clamp element disposed between the seating portion and the base portion, and configured to translate between the seating portion and the base portion;
a compressor plate coupled to a base side of the clamp element; and
a biasing element operatively coupled to the compressor plate and the base portion and configured to bias the compressor plate toward the seating portion, wherein biasing the compressor plate toward the seating portion causes the clamp element to translate to a closed configuration that prevents the elongate member from sliding axially through the catheter.

15. The system of claim 14, wherein the deployment assembly further comprises a retention element disposed upon at least one of the clamp element, the compressor plate, and the biasing element, wherein the retention element is couplable to a tensile element, wherein an actuation of the tensile element counteracts a biasing force of the biasing element and causes the clamp element to translate to an open configuration that allows the elongate member to slide through the catheter.

16. The system of claim 14, wherein the seating portion is configured to seat two elongate members.

17. The system of claim 16, wherein at least one of the two elongate members comprises a microcatheter.

18. The system of claim 14, wherein the biasing element comprises a compression spring.

19. A method for controlling an elongate member during a medical procedure, comprising:
advancing a catheter through a vessel toward a treatment area, wherein the catheter includes:
the elongate member; and
a deployment assembly positioned on a distal end of the catheter, wherein the deployment assembly is configured in a closed configuration, wherein the elongate member is restricted from translating axially through the catheter when the deployment assembly is configured in the closed configuration;
maintaining the catheter at a position adjacent to the treatment area;
actuating a tensile element, wherein actuating the tensile element counteracts a biasing force of a biasing element in the deployment assembly and causes the deployment assembly to transition from the closed configuration to an open configuration, wherein the elongate member is configured to translate axially through the catheter when the deployment assembly is configured in the open configuration; and
translating the elongate member relative to the catheter.

20. The method of claim 19, further comprising:
releasing the tensile element, wherein releasing the tensile element causes the deployment assembly to transition from the open configuration to the closed configuration; and
removing the catheter from the vessel.
